(19) United States
(12) Patent Application Publication
(10) Pub. No.: US 2026/0351385 A1
(43) Pub. Date: Nov. 5, 2026

(54) METHODS TO SUPPORT SOLUTION FOCUSED BRIEF THERAPY

(71) Applicant: Sequelae, Inc., Minneapolis, MN (US)

(72) Inventors: Shane Hoversten, Brooklyn Park, MN (US); James LaBelle, Encinitas, CA (US); Steven J. Catani, Athens, GA (US)

(73) Assignee: Sequelae, Inc., Minneapolis, MN (US)

(21) Appl. No.: 19/662,602

(22) Filed: Apr. 29, 2026

Related U.S. Application Data
(60) Provisional application No. 63/798,212, filed on May 1, 2025.

(51) Int. Cl. G16H 20/70; G16Y 10/60; G16Y 10/75

(52) U.S. Cl. CPC G16H 20/70; G16Y 10/60; G16Y 10/75

(57) ABSTRACT

Methods to support solution focused brief therapy are provided. A digital communication network (DCN) for members of the population to communicate with each other is provided. At least one question is transmitted to the individual member of the population and a request for the individual member to answer the at least one question. At least one answer is received to the at least one question from the individual member. The at least one answer is analyzed to determine the at least one value held by the individual member and a message is transmitted to the individual member based on the at least one value determined by the analysis of the at least one answer.

### CROSS REFERENCE TO RELATED APPLICATIONS

This application claims the benefit of U.S. Provisional Application No. 63/798,212, filed on May 1, 2025, which application is incorporated herein by reference in its entirety.

### BACKGROUND

Various embodiments relate generally to health care systems, methods, devices, and computer programs, and more specifically, relate to identifying at least one value or generating a narrative for an individual member based on answers to one or more questions transmitted to the individual member.

This section is intended to provide a background or context. The description may include concepts that may be pursued, but have not necessarily been previously conceived or pursued. Unless indicated otherwise, what is described in this section is not deemed prior art to the description and claims and is not admitted to be prior art by inclusion in this section.

Computers have changed the way people interact. Digital networks, which may include the use of social media, allow individuals to interact with others online and connect many people within a community. The online community can be utilized to help build positive behaviors and encourage people to make improvements in their lives.

What is needed is a way to build upon the use of digital communication networks in order to encourage individuals to engage in positive and sustained behavior changes.

### SUMMARY

### Example aspects of the present disclosure include

A method for identifying at least one value of an individual member of a population according to at least one embodiment of the present disclosure comprises providing a digital communication network (DCN) for members of the population to communicate with each other; transmitting at least one question to the individual member of the population and a request for the individual member to answer the at least one question, wherein the at least one question does not solicit the individual member to provide the at least one value; receiving at least one answer to the at least one question from the individual member; automatically analyzing the at least one answer to determine the at least one value held by the individual member; and transmitting a message to the individual member based on the at least one value determined by the analysis of the at least one answer.

Any of the aspects herein, wherein the at least one question and the at least one answer are transmitted and received using at least one of asynchronous communication and synchronous communication with a conversation bot.

Any of the aspects herein, wherein the at least one question comprises a plurality of questions.

Any of the aspects herein, wherein the at least one question and the at least one answer are transmitted and received over a predetermined time period.

Any of the aspects herein, wherein the time period is at least one of an hour, a day, a week, a month, and more than one month.

Any of the aspects herein, wherein the at least one answer received is an audio file and wherein, the audio file is transcribed automatically to text.

Any of the aspects herein, wherein the at least one answer is analyzed using a large language model to assess the at least one value contained within the answer.

Any of the aspects herein, wherein the at least one value comprises at least one of trust, interpersonal harmony, care for long-term outcomes, and family continuity.

Any of the aspects herein, wherein the at least one value determined is communicated to the individual member through the DCN.

Any of the aspects herein, wherein a request to reflect on the communicated values is transmitted to the individual member.

Any of the aspects herein, wherein the request to reflect is communicated to the individual member by a conversation bot.

Any of the aspects herein, wherein the message comprises at least one of a suggestion of a behavior change that matches the at least one value or a post is posted in the DCN related to a behavior change that matches the at least one value.

Any of the aspects herein, wherein the individual member can modify the at least one value determined by the analysis.

Any of the aspects herein, wherein the communication between the members of the population is at least one of one member to one member, one member to many members, one member to a system, the system to one member, and the system to many members.

Any of the aspects herein, wherein the system is an artificial intelligence bot that derives its communications from at least one of an analysis of communications in the DCN or biometrics provided to the DCN.

Any of the aspects herein, wherein the method is used to understand a motivation for a change in an individual member.

Any of the aspects herein, wherein the method is used to manage a chronic disease or lower a cost of health care in the population.

Any of the aspects herein, wherein the method is used to slow the progression of an adverse health condition in the individual member.

Any of the aspects herein, wherein the message comprises a communication aligned with an adverse condition associated with the individual member.

Any of the aspects herein, wherein the adverse condition is determined by automatically analyzing communications from the individual member in the DCN.

Any of the aspects herein, wherein the adverse condition comprises at least one of chronic systemic inflammation, malaise, low energy, a disease, a health risk, social dysfunction, or a prodromal disease.

Any of the aspects herein, wherein the disease comprises at least one of obesity, diabetes, rheumatoid arthritis, Crohn’s disease, psoriasis, eczema, cardiovascular disease, congestive heart failure, chronic obstructive pulmonary disease, asthma, depression, or anxiety.

Any of the aspects herein, wherein the health risk is at least one of falling or becoming infected with an illness.

A method for generating a narrative for an individual member of a population according to at least one embodiment of the present disclosure comprises providing a digital communication network (DCN) for members of the population to communicate with each other; transmitting at least one question to the individual member of the population and a request for the individual member to answer the at least one question, wherein the at least one question does not solicit the individual member to provide a narrative of the individual member’s history, aspiration, and at least one value; receiving at least one answer to the at least one question from the individual member; automatically analyzing the at least one answer to generate the narrative about the individual member; and transmitting a message to the individual member based on the narrative determined by the analysis of the at least one answer.

Any of the aspects herein, wherein the individual member is the protagonist of the narrative.

Any of the aspects herein, wherein the narrative comprises elements that reflect at least one of the individual’s life state, current lifestyle, the journey that created the narrative, the individual member’s aspirations for the future, and the individual member’s values.

Any of the aspects herein, further comprising communicating the narrative to the individual member through the DCN.

Any of the aspects herein, further comprising transmitting a request to the individual member to reflect on the narrative.

Any of the aspects herein, further comprising receiving a modification to the narrative from the individual member.

Any of the aspects herein, further comprising sharing the narrative on the DCN.

Any of the aspects herein, wherein the method is used to slow the progression of an adverse health condition of the individual member.

Any of the aspects herein, wherein a plot of the narrative comprises a theme of overcoming a challenge.

Any of the aspects herein, wherein the message comprises at least one of a suggestion of a behavior change that matches the at least one value or a post is posted in the DCN related to a behavior change that matches the at least one value.

Any of the aspects herein, wherein the message comprises a request to the individual member to reflect on at least one of their lifestyle, adverse health condition, environment, and combinations thereof.

Any of the aspects herein, wherein the method is used to manage a chronic disease or lower a cost of health care in the population.

Any of the aspects herein, further comprising determining at least one value from the narrative, and wherein the message is based on the narrative and the at least one value.

A method to optimize at least one question for an individual member according to at least one embodiment of the present disclosure comprises providing a digital communication network (DCN) for members of the population to communicate with each other; transmitting at least one question to the individual member of the population and a request for the individual member to answer the at least one question; receiving at least one answer to the at least one question from the individual member; automatically analyzing the at least one answer to determine if the at least one answer meets a suitability threshold; and repeating transmitting the at least one question, receiving the at least one answer, and automatically analyzing the at least one answer when the at least one answer does not meet the suitability threshold, wherein the suitability threshold corresponds to measures of at least one of a future outlook, concreteness of a view point, emotional valence, and combinations thereof.

Any of the aspects herein, wherein a combination of all of the at least one answers received are used to determine a suitability of the at least one question.

Any of the aspects herein, wherein the at least one question comprises a plurality of questions.

Any of the aspects herein, wherein the at least one question is selected from a pool of questions designed to illicit a suitable response.

Any of the aspects herein, wherein one or more attributes of a subsequent question is different to one or more attributes of the at least one question.

Any of the aspects herein, wherein the plurality of questions are asked in a single session.

Any of the aspects herein, wherein the plurality of questions are asked in a plurality of separate sessions, and wherein each session is separated by a period of time.

Any of the aspects herein, wherein the period of time is at least one of an hour, a day, or a week.

Any of the aspects herein, further comprising requesting that the individual member reflect on the at least one answer prior to a subsequent session.

Any of the aspects herein, wherein the at least one question that meets the suitability threshold are used to determine at least value of the individual member.

Any of the aspects herein, wherein the at least one question that meets the suitability threshold are used to generate a narrative about at least one of the individual member’s life state, current lifestyle, the journey that created the narrative, aspirations for the future, and at least one value.

Any of the aspects herein, wherein the at least one question and the at least one answer are transmitted and received at least one of verbally or in text.

A method for optimizing one or more questions according to at least one embodiment of the present disclosure comprises providing a digital communication network (DCN) for members of the population to communicate with each other; transmitting at least one question to the individual member of the population and a request for the individual member to answer the at least one question; receiving at least one answer to the at least one question from the individual member; analyzing the at least one answer to generate an outcome; and transmitting a message to the individual member based on the outcome, wherein the outcome comprises at least one of the individual member’s values, future aspirations, or a narrative.

Any of the aspects herein, further comprising: transmitting at least one subsequent question; receiving at least one subsequent answer; analyzing the at least one subsequent answer to determine a subsequent outcome; measuring a similarity between the outcome and the subsequent outcome; and repeating transmitting the at least one subsequent question, receiving the at least one subsequent answer, determining the subsequent outcome, and measuring the similarity when the similarity is below a similarity threshold, wherein the similarity threshold assess a difference between one or more parameters of the outcome and one or more parameters of the subsequent outcome.

Any of the aspects herein, wherein a combination of all of the at least one answers received are used to generate the outcome.

Any of the aspects herein, wherein the at least one question is asked before the outcome is generated.

Any of the aspects herein, wherein similarity is requested for two subsequent question of the at least one subsequent question to satisfy the similarity threshold.

A method for inducing reflection in an individual member of a population according to at least one embodiment of the present disclosure comprises providing a digital communication network (DCN) for members of the population to communicate with each other; transmitting at least one question to the individual member of the population and a request for the individual member to answer the at least one question; receiving at least one answer to the at least one question from the individual member; and automatically analyzing the at least one answer to determine an emotional valence of the individual member, wherein a subsequent question is based in part on increasing the emotional valence of subsequent answers.

Any aspect in combination with any one or more other aspects.

Any one or more of the features disclosed herein.

Any one or more of the features as substantially disclosed herein.

Any one or more of the features as substantially disclosed herein in combination with any one or more other features as substantially disclosed herein.

Any one of the aspects/features/embodiments in combination with any one or more other aspects/features/embodiments.

Use of any one or more of the aspects or features as disclosed herein.

It is to be appreciated that any feature described herein can be claimed in combination with any other feature(s) as described herein, regardless of whether the features come from the same described embodiment.

### BRIEF DESCRIPTION OF THE DRAWINGS

Aspects of the described embodiments are more evident in the following description, when read in conjunction with the attached Figures.

FIG. 1 shows a simplified block diagram of devices, in accordance with one or more embodiments.

FIG. 2 is a logic flow diagram that illustrates the operation of a method, in accordance with one or more embodiments.

FIG. 3 is a logic flow diagram that illustrates the operation of a method, in accordance with one or more embodiments.

FIG. 4 is a logic flow diagram that illustrates the operation of a method, in accordance with one or more embodiments.

FIG. 5 is a logic flow diagram that illustrates the operation of a method, in accordance with one or more embodiments.

FIG. 6 is a logic flow diagram that illustrates the operation of a method, in accordance with one or more embodiments.

FIG. 7A shows an example of a computing system, in accordance with one or more embodiments.

FIG. 7B shows an example of a network, in accordance with one or more embodiments.

### DETAILED DESCRIPTION

Various embodiments are directed to providing solutions-based therapy based on answers received from an individual member in response to questions presented to the individual member within a digital communication network (DCN). The solutions-based therapy may be based in part on creating greater awareness of lifestyle changes and medial options within the DCN.

Online communities can be provided so that people can learn about healthy lifestyle practices and work to improve their health between clinical touchpoints such as office visits (whether through communications from an influential person or from other members). To help incentivize healthy behaviors, patients can use tools like in-home tests and biosensors that measure how well their health actions are working.

In combination with the online communities, solutions-based therapy is a simple and effective method to help individuals achieve the motivation to engage successfully in sustained behavior change. Solutions-based therapy has been shown useful for a wide range of conditions, such as, for example, depression, obesity, etc. and in both one-on-one and group sessions. However, conventionally provided solutions-based therapy is generally not broadly scalable.

Thus, it is desirable to provide methods that can be used in a community where identified values, a narrative, an outcome, messages based on the values, narrative, or outcome can be sent to a member based on answers provided by the member in response to questions sent to the member. The values, narratives, outcomes, or messages can be used to promote long-term behavior change. Further it is desirable to optimize the questions provided to the member to illicit or induce answers indicative of long-term change in the member. Such methods as will be described in detail below are scalable and provide new uses related to finding and activating motivation in individuals to engage in life-improving behavior change.

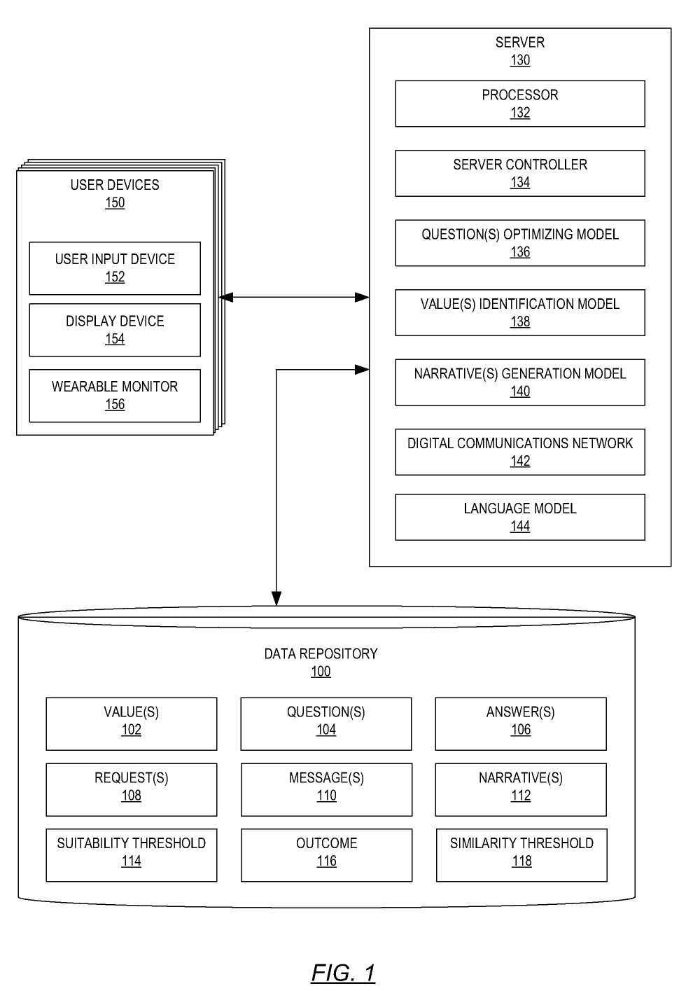

The system shown in FIG. 1 includes a data repository (100). The data repository (100) is a type of storage unit or device (e.g., a file system, database, data structure, or any other storage mechanism) for storing data (described below). The data repository (100) may include multiple different, potentially heterogeneous, storage units and/or devices.

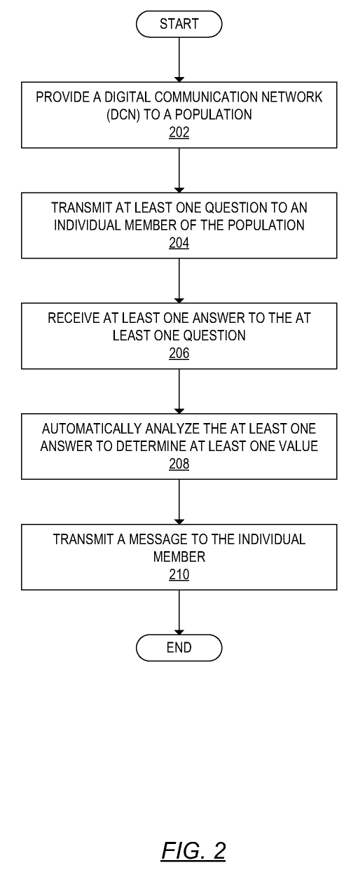

The data repository (100) stores value(s) (102). The values (102) correspond to one or more values held by an individual member of a population. The one or more values (102) may be, for example, trust, interpersonal harmony, care for long-term outcomes, family continuity, etc. The values (102) can be determined based on the individual’s answers to one or more questions (104) as will be described in more detail in FIG. 2.

The data repository (100) also stores question(s) (104). The questions (104) are questions directed to the individual member and may include yes/no questions or open-ended questions. The questions (104) may be designed so as to not solicit the individual member to provide the values (102), a narrative (112), and/or aspects of the narrative (112) (e.g., the individual member’s history or aspirations). The questions (104) can include one question, two questions, or more than two questions. In some embodiments, the questions (104) used for generating a narrative (112) can be based on different archetypes, such as, for example, the miracle, the struggle, etc.

The data repository (100) also stores request(s) (108). The request (108) may be, for example, a request (108) for the individual member to answer the question (104). The request may include buttons (e.g., yes/no buttons), a text and/or media box for the individual member to type in or attach media.

The data repository (100) also stores answer(s) (106). The answers (106) are answers (106) to the questions (104) and the requests (108) received from the individual member. The answers (106) may include, for example, a yes/no answer, text, and/or media (e.g., audio, visual, images, videos, etc.). As will be described in detail in FIGS. 2, 3, 4, 5, and 6, the answers (106) may be used to identify the values (102), generate the narrative (112), determine an outcome (116), and/or optimize the questions (104).

The data repository (100) also stores message(s) (110). The messages (110) are generated and transmitted to the individual member after the answer(s) (106) are received from the individual member. The messages (110) may be based on, for example, the values (102) identified, the narrative (112) generated, or an outcome (116) determined from the answers (106). The messages (110) may include, for example, one or more suggestions for the individual member to incorporate into their life and may include suggestions directed to behavior modification.

The data repository (100) also stores narrative(s) (112). The narrative (112) may be generated based on the answer(s) (106) received from the individual member. The narrative (112) may correlate to or represent the individual member’s life state, the journey that created the life state, and the individual member’s aspirations for the future that are useful in creating and sustaining motivation to make health inducing behavior change in the individual member. The narrative (112) may include, for example, elements that reflect the individual member’s life state, current lifestyle, the journey that created the life state, the individual member’s aspirations for the future, and/or the values (102). In some embodiments, the individual member is the protagonist or hero of the narrative (112). In other embodiments, the narrative (112) includes a plot that involves overcoming a struggle.

The data repository (100) also stores suitability threshold(s) (114). The answers (106) provided by the individual member may be compared to the suitability threshold (114) to determine if the question (104) asked to illicit the answer (106) was suitable. The suitable threshold (114) may include measures, such as future outlook, concreteness of view-point, emotional valence, or combinations thereof.

The data repository (100) also stores outcome(s) (116). The outcomes (116) may include variables, such as the individual member’s values, narrative, or future aspirations. The outcome (116) may be determined from the answers (106) provided by the individual member. The outcome (116) may be used to generate the messages (110).

The data repository (100) also stores similarity threshold(s) (118). The similarity threshold (118) may be used to measure or assess the differences between the parameters associated with the outcome(s) (118). For example, the similarity threshold (118) can be used to determine whether a first outcome and a second or subsequent outcome (116) are similar. A similarity between the first outcome and the second outcome may be measured or quantified and the similarity may be compared to the similarity threshold (118).

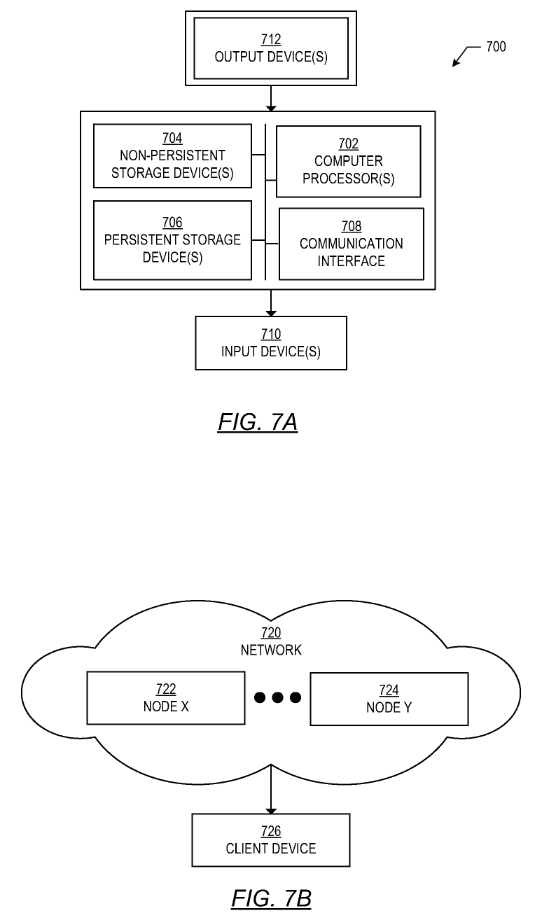

The system shown in FIG. 1 may include other components. For example, the system shown in FIG. 1 also may include a server (130). The server (130) is one or more computer processors, data repositories, communication devices, and supporting hardware and software. The server (130) may be in a distributed computing environment. The server (130) is configured to execute one or more applications, such as a question(s) optimizing model (136), a value(s) identification model (138), and/or a narrative(s) generation model (140). An example of a computer system and network that may form the server (130) is described with respect to FIGS. 7A and 7B.

The server (130) also includes a computer processor (132). The computer processor (132) is one or more hardware or virtual processors which may execute computer readable program code that defines one or more applications, such as the question(s) optimizing model (136), the value(s) identification model (138), and/or the narrative(s) generation model (140). An example of the computer processor (132) is described with respect to the computer processor(s) (702) of FIG. 7A.

The server (130) also may include a server controller (134). The server controller (134) is software or application specific hardware which, when executed by the computer processor (132), controls and coordinates operation of the software or application specific hardware described herein. Thus, the sever controller (138) may control and coordinate execution of the question(s) optimizing model (136), the value(s) identification model (138), and/or the narrative(s) generation model (140).

The server (130) also may include the questions optimizing model (136). The questions optimizing model (136) is software or application specific hardware which, when executed by the computer processor (132) receives at least the questions (104) and the answers (106) as input to determine if the questions (104) have been optimized to produce the desired answers (106). The desired answers (106) may, for example, indicate that the individual member has or is undertaking positive behavior modification or lifestyle changes.

The server (130) also may include the values identification model (138). The values identification model (138) is software or application specific hardware which, when executed by the computer processor (132) receives the answers (106) as input to determine or identify values (102) in the answers (106). In some embodiments, the values identification model (138) may also receive the questions (104) as input.

The server (130) also may include the narratives generation model (140). The narratives generation model (140) is software or application specific hardware which, when executed by the computer processor (132) receives the answers (106) as input to determine or identify values (102) in the answers (106). In some embodiments, the narratives generation model (140) may also receive the questions (104) as input.

The server (130) also includes a digital communications network (DCN) (142). The DCN is a network through which members of a population can interact with each other, or with a system supported by the DCN. For example, an individual member can interact with the DCN (142) by receiving questions (104) and a request (108) to answer the questions (104) and submitting their answer (106) to the questions (104) to the DCN (142). The DCN (142) can use the answer(s) (106) for a variety of purposes, such as, for example, determining the values (102) of the individual member, generating the narrative (112) for the individual member, optimizing the questions (104) asked, and/or determining the outcome (116).

The DCN (142) can also provide means for members of the population to communicate with each other. For example, the individual member can share their values (102), narrative (112), outcome (116), or any other communication with other members in the DCN (142). Communications between the members of the population in the DCN (142) can be, for example, one to one, one to many, one to the system, the system to one, or the system to many. Further, in some embodiments, the DCN (142) may use artificial intelligence (AI) to derive communications from the DCN (142) from an analysis of communications in the DCN (142) or from biometrics provided to the DCN (142) from the members.

The server (130) also includes a language model (144). The language model (144) is a natural language processing machine learning model. An example of the language model (144) may be a large language model, such as CHATGPT^{®} or LLAMA^{®}. However, many different language models may be used. In some embodiments, the language model (144) can be used to assess the values (102) contained within an answer (106). In other embodiments, the language model (144) can be used to generate the narrative (112).

The system shown in FIG. 1 also may include one or more user devices (150). The user devices (150) may be considered remote or local. A remote user device is a device operated by a third-party (e.g., an end user of a chatbot) that does not control or operate the system of FIG. 1. Similarly, the organization that controls the other elements of the system of FIG. 1 may not control or operate the remote user device. Thus, a remote user device may not be considered part of the system of FIG. 1.

In contrast, a local user device is a device operated under the control of the organization that controls the other components of the system of FIG. 1. Thus, a local user device may be considered part of the system of FIG. 1.

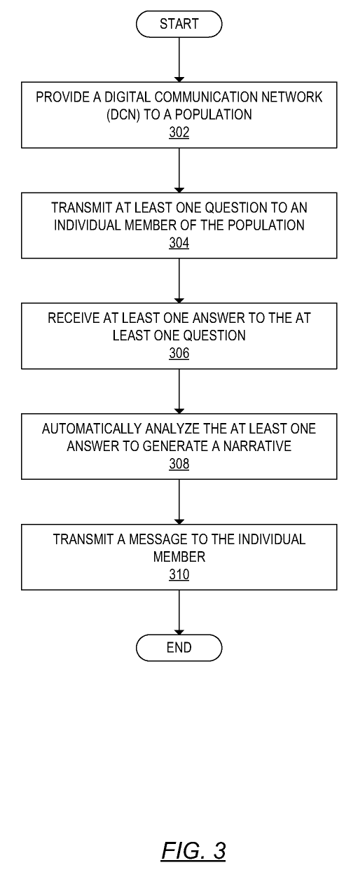

In any case, the user devices (150) are computing systems (e.g., the computing system (300) shown in FIG. 3A) that communicate with the server (130). The user devices (150) may include a wearable monitor (156) and be configured to send stress indicator data (110) to the server (130). In an alternative embodiment, a separate wearable device may be in communication with the user device (150), such as a smart watch, or blood pressure monitor. The user devices (150) may also include a user input device (152) and/or a display device (154).

In contrast, a local user device is a device operated under the control of the organization that controls the other components of the system of FIG. 1. Thus, a local user device may be considered part of the system of FIG. 1.

While FIG. 1 shows a configuration of components, other configurations may be used without departing from the scope of one or more embodiments. For example, various components may be combined to create a single component. As another example, the functionality performed by a single component may be performed by two or more components.

FIG. 2 is a logic flow diagram that illustrates a method, and a result of execution of computer program instructions, in accordance with various embodiments. The method can be used to identify one or more values of an individual member. The one or more values can then be used, in some embodiments, to generate messages based on the one or more values that include suggestions or recommendations for encouraging and sustaining a behavior change in the individual member. In other embodiments, the method can be used to slow the progression of an adverse health condition in an individual member of a population. In still other embodiments, the method can be used to understand a motivation for change in an individual member. In still other embodiments, the method can be used to manage a chronic disease or lower a cost of health care in the population.

At Block 202, a step of providing a digital communication network (DCN) to a population is provided. The DCN may be the same as or similar to the DCN (142). As previously described, members of the population enrolled in the DCN can communicate with each other or with the DCN itself.

At Block 204, a step of transmitting at least one question to an individual member of the population is provided. The at least one question may be the same as or similar to the question (104). The question may be transmitted to, for example, a user device such as the user device (150) by the DCN. The question may be asked using asynchronous communication or in a synchronous communication with a conversation bot.

At Block 206, a step of receiving at least one answer to the at least one question is provided. The at least one answer may be the same as or similar to the answer (106). The question may be transmitted by, for example, the user device to the DCN. The answer may be answered using asynchronous communication or in a synchronous communication with a conversation bot.

The question and the answer may be asked and answered, respectively, over a defined time period. In some embodiments, the time period is an hour, a day, a week, a month, several months, or any other increment of time. The question and/or the answer may also be provided verbally and any verbal question or answer may be transcribed automatically to text.

At Block 208, a step of automatically analyzing the at least one answer to determine at least one value is provided. The at least one value may be the same as or similar to the value(s) (102) and may be determined by a values identification model such as the values identification model (138) using the answer as input. The value(s), once identified, may be communicated to the individual member via the DCN. In some embodiments, the individual member can modify the identified values.

At Block 210, a step of transmitting a message to the individual member is provided. The message may be based on the identified value(s). For example, the message may be a request to the individual member to reflect on the value(s), such as, for example, their lifestyle, adverse health condition, environment, and/or combinations thereof. In such examples, the reflection may be facilitated by a conversation bot. In other examples or embodiments, the message to the individual member may be a suggestion of a behavior change that matches the individual member’s identified values.

In some embodiments the message is directly communicated to the individual member. In other embodiments, the message is a post in the community via the DCN related to a behavior change aligned with the individual member’s identified value. In still other embodiments, the message is aligned with an adverse condition associated with the individual member. In such embodiments, the adverse condition may be determined from an analysis of the individual member’s communications in the DCN. The adverse condition may be, for example, chronic systemic inflammation (CSI); malaise or low-energy, and/or a disease. The disease may be a prodromal disease, obesity, diabetes, rheumatoid arthritis, Crohn’s, psoriasis, eczema, cardiovascular disease, congestive heart failure, chronic obstructive pulmonary disease, asthma, depression, or anxiety. In other cases, the condition may be a health risk, such as falling or becoming infected. In some cases, the condition may be a social dysfunction or a prodromal disease.

FIG. 3 is a logic flow diagram that illustrates a method, and a result of execution of computer program instructions, in accordance with various embodiments. The method can be used to generate a narrative of an individual member. The narrative can then be, in some embodiments, transmitted to the individual member and used to encourage sustainable behavioral changes. In some embodiments, the method described below can be used in slowing the progression of an adverse health condition. In other embodiments, the method described below can be used to manage a chronic disease or lower a cost of healthcare in the population.

At Block 302, a step of providing a digital communication network (DCN) to a population is provided. The Block 302 is generally the same as or similar to the Block 202 of FIG. 2 described above.

At Block 304, a step of transmitting at least one question to an individual member of the population is provided. The Block 304 is generally the same as or similar to the Block 204 of FIG. 2 described above.

At Block 306, a step of receiving at least one answer to the at least one question is provided. The Block 306 is generally the same as or similar to the Block 206 of FIG. 2 described above.

At Block 308, a step of automatically analyzing the at least one answer to generate a narrative is provided. The narrative may be the same as or similar to the narrative (112). The narrative may, in some embodiments, be transmitted to the individual member via the DCN. In such embodiments, the individual member may be requested to reflect on the narrative. Such request may be facilitated by, for example, a conversation bot. In the same embodiments, the individual member may be able to modify the narrative. Further, the narrative can be shared in the DCN with, for example, the population or a subset of members.

In some embodiments, one or more values such as the values (102) may also be identified during the analysis of the at least one answer. In such embodiments, the method described herein may be used with the narrative to generate motivational communications or messages to the individual member. In other words, the method can be used to create motivation to ultimately slow disease progression, manage a chronic disease, or lower healthcare costs.

At Block 310, a step of transmitting a message to the individual member is provided. The message may be the same as or similar to the message (110). The message may be, for example, a suggestion of a behavior change that matches the individual member’s values (that are also provided or used to generate the narrative). The message may also be a post in the DCN related to a behavior change aligned with the individual member’s values. The message may also be a request to the individual member to reflect on their lifestyle, adverse health condition, environment, or combinations thereof in light of the narrative. Such request may be facilitated by a conversation bot.

The message may also be aligned with an adverse condition associate with the individual member. As previously described, the adverse condition may be, for example, chronic systemic inflammation (CSI) malaise or low-energy, and/or a disease. The disease may be a prodromal disease, obesity, diabetes, rheumatoid arthritis, Crohn’s, psoriasis, eczema, cardiovascular disease, congestive heart failure, chronic obstructive pulmonary disease, asthma, depression, or anxiety. In other cases, the condition may be a health risk, such as falling or becoming infected. In some cases, the condition may be a social dysfunction or a prodromal disease.

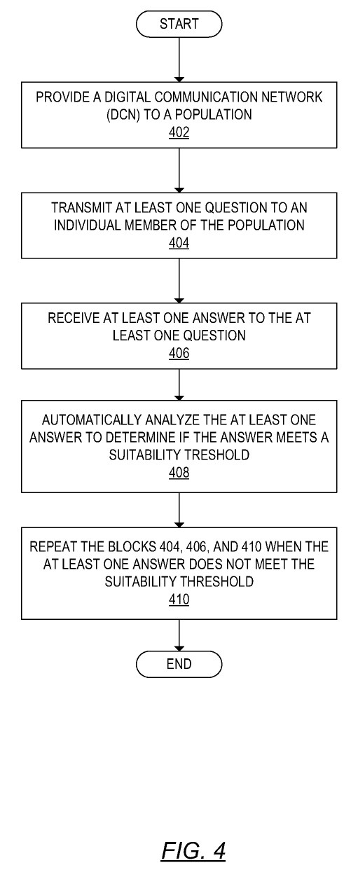

FIG. 4 is a logic flow diagram that illustrates a method, and a result of execution of computer program instructions, in accordance with various embodiments. The method can be used to optimize one or more questions that are transmitted to an individual member. The method can also be used to slow the progression of an adverse condition, manage a disease, or reduce healthcare costs of a population.

At Block 402, a step of providing a digital communication network (DCN) to a population is provided. The Block 402 is generally the same as or similar to the Block 202 of FIG. 2 described above.

At Block 404, a step of transmitting at least one question to an individual member of the population is provided. The Block 404 is generally the same as or similar to the Block 204 of FIG. 2 described above. In the Block 404, additionally or alternatively, the question(s) may be selected from a pool of questions designed to illicit a suitable response. In some embodiments, several questions may be asked before a first suitability analysis.

At Block 406, a step of receiving at least one answer to the at least one question is provided. The Block 406 is generally the same as or similar to the Block 206 of FIG. 2 described above.

At Block 408, a step of automatically analyzing the at least one answer to determine if the answer meets a suitability threshold is provided. The suitability threshold may be the same as or similar to the suitability threshold (114). The suitability threshold correlates to a measurement of the individual member’s future outlook, concreteness of a viewpoint, emotional valence, or combinations thereof. Whether the answer meets the suitability threshold or not can be used to optimize the question(s) asked to the individual member.

In some embodiments, the suitability can be based on a combined response of all answers (when more than one answer is received). In other embodiments, multiple questions can be asked before a suitability test (and in some embodiments, before a first suitability test).

In some embodiments, answers that are deemed suitable can be used to determine the individual’s values. In other embodiments, answers that are deemed suitable can also be used to create a narrative, such as the narrative (112) about the individual member’s life state, current lifestyle, the journey that created it, their aspirations for the future, and/or their values.

At Block 410, the Blocks 404, 406, and 410 are repeated when the at least one answer does not meet the suitability threshold. When subsequent questions are asked, attributes of the subsequent questions may be different to attributes of previously asked questions. The subsequent question can be asked in a separate session, separated by a time period from the questions in the Block 404. The time period may be, for example, an hour, a day, a week, or any length of time.

In some embodiments, the combined responses of all answers are used to determine suitability. In some embodiments, the individual member may be requested to reflect on their answer before the next set of questions are asked.

The method described in FIG. 4 can also include analyzing the answers to create an outcome such as the outcome (116), if the answers meet the suitability threshold. The method may also include sending the individual member a message such as the message (110), based on the outcome. The message may be aligned with an adverse condition associated with the individual member. As previously described, the adverse condition may be, for example, chronic systemic inflammation (CSI), malaise or low-energy, and/or a disease. The disease may be a prodromal disease, obesity, diabetes, rheumatoid arthritis, Crohn’s, psoriasis, eczema, cardiovascular disease, congestive heart failure, chronic obstructive pulmonary disease, asthma, depression, or anxiety. In other cases, the condition may be a health risk, such as falling or becoming infected. In some cases, the condition may be a social dysfunction or a prodromal disease.

The method described in FIG. 4 can also include analyzing the answers to determine one or more values such as the values (102), if the answers meet the suitability threshold. The method may also include generating a narrative such as the narrative (112), based on the determined values. The values may also be communicated to the individual member via the DCN and the individual member may be requested to reflect on the communicated values. Such reflection may be facilitated by a conversation bot. The individual member can also modify the determined values.

When the narrative is generated, the method described in FIG. 4 can also include communicating the narrative to the individual member via the DCN. The individual member may be requested to reflect on the narrative and such request may be facilitated by a conversation bot. In some instances, the individual member can modify the narrative.

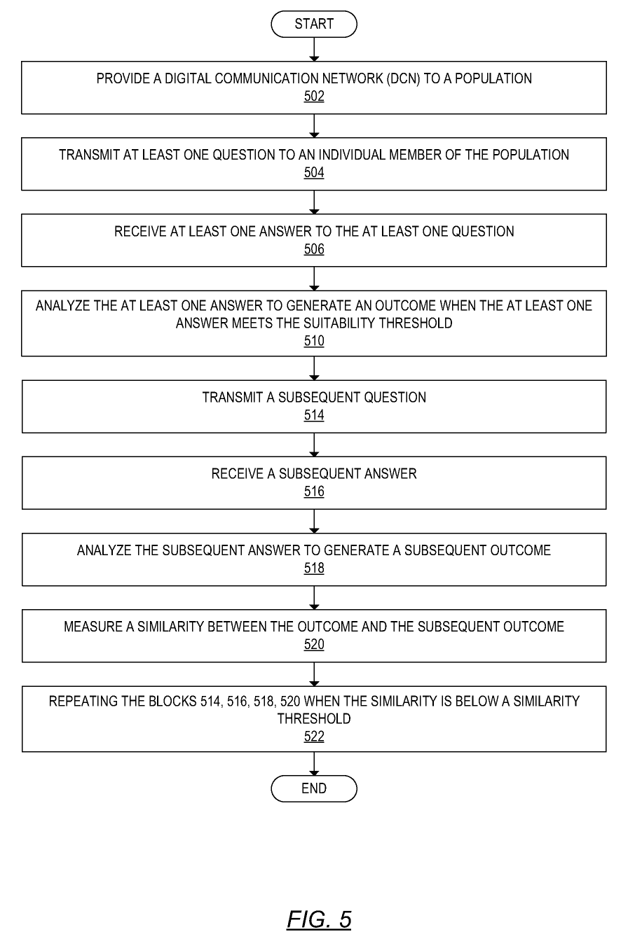

FIG. 5 is a logic flow diagram that illustrates a method, and a result of execution of computer program instructions, in accordance with various embodiments. The method can also be used to optimize one or more questions that are transmitted to an individual member. More specifically, the stability of the resultant analysis of outcomes itself is used to optimize the questions.

At Block 502, a step of providing a digital communication network (DCN) to a population is provided. The Block 502 is generally the same as or similar to the Block 202 of FIG. 2 described above.

At Block 504, a step of transmitting at least one question to an individual member of the population is provided. The Block 504 is generally the same as or similar to the Block 204 of FIG. 2 described above. The at least one question, in some instances, may be selected from a pool of questions designed to illicit a suitable response.

At Block 506, a step of receiving at least one answer to the at least one question is provided. The Block 506 is generally the same as or similar to the Block 206 of FIG. 2 described above.

At Block 510, a step of analyzing the at least one answer to generate an outcome is provided. The outcome may be the same as or similar to the outcome (116). In some embodiments, multiple questions may be asked prior to determining the outcome. In such embodiments, the combined responses to all of the answers are used to determine the outcome. Prior to Block 514 below, the individual member may be requested to reflect on the at least one answer they provided.

At Block 514, a step of transmitting a subsequent question is provided. The subsequent question can be asked in a separate session, separated by a time period from the questions in the Block 504. The time period may be, for example, an hour, a day, a week, or any length of time. The attributes of the subsequent question may be different than attributes of the question transmitted in the Block 504.

At Block 516, a step of receiving a subsequent answer is provided. The Block 516 may be the same as or similar to the Block 206 of FIG. 2 described above.

At Block 518, a step of analyzing the subsequent answer to generate a subsequent outcome is provided. The Block 518 may be the same as or similar to the Block 510 described above.

At Block 520, a step of measuring a similarity between the outcome and the subsequent outcome is provided. Measuring the similarity assesses a difference between parameters associated with the outcome and the subsequent outcome.

At Block 522, a step of repeating the Blocks 514, 516, 518, 520 when the similarity is below a similarity threshold is provided. The similarity threshold may be the same as or similar to the similarity threshold (118). In some embodiments, similarity between two subsequent questions are required to also satisfy the similarity threshold.

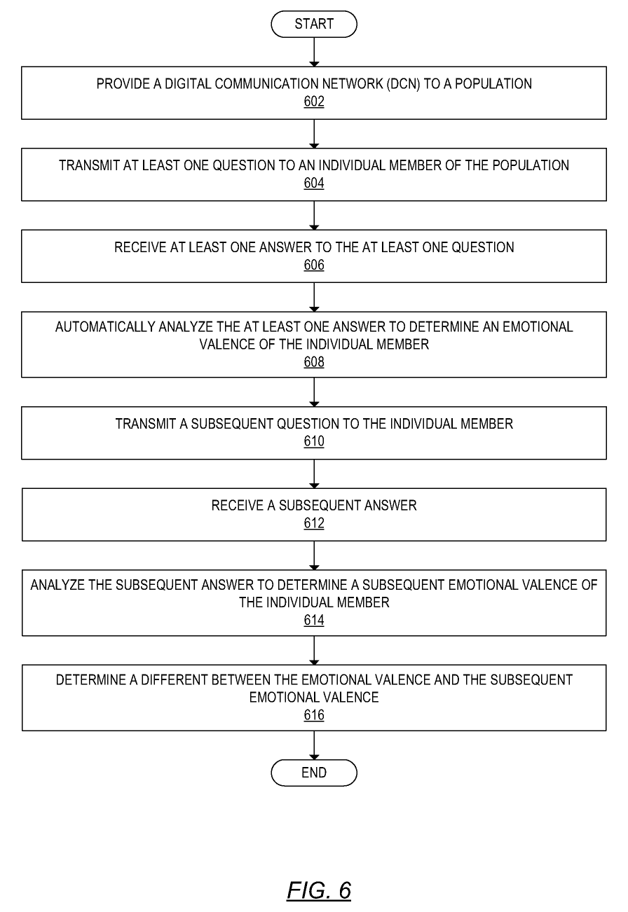

FIG. 6 is a logic flow diagram that illustrates a method, and a result of execution of computer program instructions, in accordance with various embodiments. Because not all types of reflections are equal, reflections that focus on an emotional component of an experience can be more effective at causing an individual member to take action to improve their lifestyle or behaviors and, for example, slow the progression of a chronic disease. Thus, the method can be used to determine an emotional valence of an individual member. The method can also be used to optimize questions such as the questions (104) using the emotional valence.

At Block 602, a step of providing a digital communication network (DCN) to a population is provided. The Block 602 is generally the same as or similar to the Block 202 of FIG. 2 described above.

At Block 604, a step of transmitting at least one question to an individual member of the population is provided. The Block 604 is generally the same as or similar to the Block 204 of FIG. 2 described above.

At Block 606, a step of receiving at least one answer to the at least one question is provided. The Block 606 is generally the same as or similar to the Block 206 of FIG. 2 described above.

At Block 608, a step of analyzing the at least one answer to determine an emotional valence of the individual member is provided.

At Block 610, a step of transmitting a subsequent question is provided.

At Block 612, a step of receiving a subsequent answer is provided.

At Block 614, a step of analyzing the subsequent answer to determine a subsequent emotional valence of the individual member is provided.

At Block 616, a step of determining a difference between the emotional valence and the subsequent emotional valence is provided. The difference can be used to determine if the emotional valence increased in subsequent answers. An increase in emotional valence is positive whereas, a decrease in emotional valence is associated with a negative feeling. The change in emotional valence can be used to, for example, optimize the questions transmitted in the Blocks 604 and 610.

One or more embodiments may be implemented on a computing system specifically designed to achieve an improved technological result. When implemented on a computing system, the features and elements of the disclosure provide a significant technological advancement over computing systems that do not implement the features and elements of the disclosure. Any combination of mobile, desktop, server, router, switch, embedded device, or other types of hardware may be improved by including the features and elements described in the disclosure.

For example, as shown in FIG. 7A, the computing system (700) may include one or more computer processor(s) (702), non-persistent storage device(s) (704), persistent storage device(s) (706), a communication interface (708) (e.g., Bluetooth interface, infrared interface, network interface, optical interface, etc.), and numerous other elements and functionalities that implement the features and elements of the disclosure. The computer processor(s) (702) may be an integrated circuit for processing instructions. The computer processor(s) (702) may be one or more cores, or micro-cores, of a processor. The computer processor(s) (702) includes one or more processors. The computer processor(s) (702) may include a central processing unit (CPU), a graphics processing unit (GPU), a tensor processing unit (TPU), combinations thereof, etc.

The input device(s) (710) may include a touchscreen, keyboard, mouse, microphone, touchpad, electronic pen, or any other type of input device. The input device(s) (710) may receive inputs from a user that are responsive to data and messages presented by the output device(s) (712). The inputs may include text input, audio input, video input, etc., which may be processed and transmitted by the computing system (700) in accordance with one or more embodiments. The communication interface (708) may include an integrated circuit for connecting the computing system (700) to a network (not shown) (e.g., a local area network (LAN), a wide area network (WAN), such as the Internet, mobile network, or any other type of network) or to another device, such as another computing device, and combinations thereof.

Further, the output device(s) (712) may include a display device, a printer, external storage, or any other output device. One or more of the output device(s) (712) may be the same or different from the input device(s) (710). The input device(s) (710) and output device(s) (712) may be locally or remotely connected to the computer processor(s) (702). Many different types of computing systems exist, and the aforementioned input device(s) (710) and output device(s) (712) may take other forms. The output device(s) (712) may display data and messages that are transmitted and received by the computing system (700). The data and messages may include text, audio, video, etc., and include the data and messages described above in the other figures of the disclosure.

Software instructions in the form of computer readable program code to perform embodiments may be stored, in whole or in part, temporarily or permanently, on a non-transitory computer readable medium, such as a solid-state drive (SSD), compact disk (CD), digital video disk (DVD), storage device, a diskette, a tape, flash memory, physical memory, or any other computer readable storage medium. Specifically, the software instructions may correspond to computer readable program code that, when executed by the computer processor(s) (702), is configured to perform one or more embodiments, which may include transmitting, receiving, presenting, and displaying data and messages described in the other figures of the disclosure.

The computing system (700) in FIG. 7A may be connected to, or be a part of, a network. For example, as shown in FIG. 7B, the network (720) may include multiple nodes (e.g., node X (722) and node Y (724), as well as extant intervening nodes between node X (722) and node Y (724)). Each node may correspond to a computing system, such as the computing system shown in FIG. 7A, or a group of nodes combined may correspond to the computing system shown in FIG. 7A. By way of an example, embodiments may be implemented on a node of a distributed system that is connected to other nodes. By way of another example, embodiments may be implemented on a distributed computing system having multiple nodes, where each portion may be located on a different node within the distributed computing system. Further, one or more elements of the aforementioned computing system (700) may be located at a remote location and connected to the other elements over a network.

The nodes (e.g., node X (722) and node Y (724)) in the network (720) may be configured to provide services for a client device (726). The services may include receiving requests and transmitting responses to the client device (726). For example, the nodes may be part of a cloud computing system. The client device (726) may be a computing system, such as the computing system shown in FIG. 7A. Further, the client device (726) may include or perform all or a portion of one or more embodiments.

The computing system of FIG. 7A may include functionality to present data (including raw data, processed data, and combinations thereof), such as results of comparisons and other processing. For example, presenting data may be accomplished through various presenting methods. Specifically, data may be presented by being displayed in a user interface, transmitted to a different computing system, and stored. The user interface may include a graphical user interface (GUI) that displays information on a display device. The GUI may include various GUI widgets that organize what data is shown, as well as how data is presented to a user. Furthermore, the GUI may present data directly to the user, e.g., data presented as actual data values through text, or rendered by the computing device into a visual representation of the data such as through visualizing a data model.

Various operations described are purely exemplary and imply no particular order. Further, the operations can be used in any sequence when appropriate and can be partially used. With the above embodiments in mind, it should be understood that additional embodiments can employ various computer-implemented operations involving data transferred or stored in computer systems. These operations are those requiring physical manipulation of physical quantities. Usually, though not necessarily, these quantities take the form of electrical, magnetic, or optical signals capable of being stored, transferred, combined, compared, and otherwise manipulated.

Any of the operations described that form part of the presently disclosed embodiments may be useful machine operations. Various embodiments also relate to a device or an apparatus for performing these operations. The apparatus can be specially constructed for the required purpose, or the apparatus can be a general-purpose computer selectively activated or configured by a computer program stored in the computer. In particular, various general-purpose machines employing one or more processors coupled to one or more computer readable medium, described below, can be used with computer programs written in accordance with the teachings herein, or it may be more convenient to construct a more specialized apparatus to perform the required operations.

The procedures, processes, and/or modules described herein may be implemented in hardware, software, embodied as a computer-readable medium having program instructions, firmware, or a combination thereof. For example, the functions described herein may be performed by a processor executing program instructions out of a memory or other storage device.

The foregoing description has been directed to particular embodiments. However, other variations and modifications may be made to the described embodiments, with the attainment of some or all of their advantages. Modifications to the above-described systems and methods may be made without departing from the concepts disclosed herein. Accordingly, the invention should not be viewed as limited by the disclosed embodiments. Furthermore, various features of the described embodiments may be used without the corresponding use of other features. Thus, this description should be read as merely illustrative of various principles, and not in limitation of the invention.

The present invention may be further exemplified by one, or a combination of one or more of, the following statements:
Statement 1. A method for identifying at least one value of an individual member of a population, the method comprising: providing a digital communication network (DCN) for members of the population to communicate with each other; transmitting at least one question to the individual member of the population and a request for the individual member to answer the at least one question, wherein the at least one question does not solicit the individual member to provide the at least one value; receiving at least one answer to the at least one question from the individual member; automatically analyzing the at least one answer to determine the at least one value held by the individual member; and transmitting a message to the individual member based on the at least one value determined by the analysis of the at least one answer.
Statement 2. The method of Statement 1, wherein the at least one question and the at least one answer are transmitted and received using at least one of asynchronous communication and synchronous communication with a conversation bot.
Statement 3. The method of Statement 1, wherein the at least one question comprises a plurality of questions.
Statement 4. The method of Statement 1, wherein the at least one question and the at least one answer are transmitted and received over a predetermined time period.
Statement 5. The method of Statement 4, wherein the time period is at least one of an hour, a day, a week, a month, and more than one month.
Statement 6. The method of Statement 1, wherein the at least one answer received is an audio file and wherein the audio file is transcribed automatically to text.
Statement 7. The method of Statement 1, wherein the at least one answer is analyzed using a large language model to assess the at least one value contained within the answer.
Statement 8. The method of Statement 1, wherein the at least one value comprises at least one of trust, interpersonal harmony, care for long-term outcomes, and family continuity.
Statement 9. The method of Statement 1, wherein the at least one value determined is communicated to the individual member through the DCN.
Statement 10. The method of Statement 9, wherein a request to reflect on the communicated values is transmitted to the individual member.
Statement 11. The method of Statement 10, wherein the request to reflect is communicated to the individual member by a conversation bot.
Statement 12. The method of Statement 1, wherein the message comprises at least one of a suggestion of a behavior change that matches the at least one value or a post is posted in the DCN related to a behavior change that matches the at least one value.
Statement 13. The method of Statement 1, wherein the individual member can modify the at least one value determined by the analysis.
Statement 14. The method of Statement 1, wherein the communication between the members of the population is at least one of one member to one member, one member to many members, one member to a system, the system to one member, and the system to many members.
Statement 15. The method of Statement 14, wherein the system is an artificial intelligence bot that derives its communications from at least one of an analysis of communications in the DCN or biometrics provided to the DCN.
Statement 16. The method of Statement 1, wherein the method is used to understand a motivation for a change in an individual member.
Statement 17. The method of Statement 1, wherein the method is used to manage a chronic disease or lower a cost of health care in the population.
Statement 18. The method of Statement 1, wherein the method is used to slow the progression of an adverse health condition in the individual member.
Statement 19. The method of Statement 1, wherein the message comprises a communication aligned with an adverse condition associated with the individual member.
Statement 20. The method of Statement 19, wherein the adverse condition is determined by automatically analyzing communications from the individual member in the DCN.
Statement 21. The method of Statement 20, wherein the adverse condition comprises at least one of chronic systemic inflammation, malaise, low energy, a disease, a health risk, social dysfunction, or a prodromal disease.
Statement 22. The method of Statement 21, wherein the disease comprises at least one of obesity, diabetes, rheumatoid arthritis, Crohn’s disease, psoriasis, eczema, cardiovascular disease, congestive heart failure, chronic obstructive pulmonary disease, asthma, depression, or anxiety.
Statement 23. The method of Statement 21, wherein the health risk is at least one of falling or becoming infected with an illness.
Statement 24. A method for generating a narrative for an individual member of a population, the method comprising: providing a digital communication network (DCN) for members of the population to communicate with each other; transmitting at least one question to the individual member of the population and a request for the individual member to answer the at least one question, wherein the at least one question does not solicit the individual member to provide a narrative of the individual member’s history, aspiration, and at least one value; receiving at least one answer to the at least one question from the individual member; automatically analyzing the at least one answer to generate the narrative about the individual member; and transmitting a message to the individual member based on the narrative determined by the analysis of the at least one answer.
Statement 25. The method of Statement 24, wherein the individual member is the protagonist of the narrative.
Statement 26. The method of Statement 24, wherein the narrative comprises elements that reflect at least one of the individual’s life state, current lifestyle, the journey that created the narrative, the individual member’s aspirations for the future, and the individual member’s values.
Statement 27. The method of Statement 24, further comprising communicating the narrative to the individual member through the DCN.
Statement 28. The method of Statement 27, further comprising transmitting a request to the individual member to reflect on the narrative.
Statement 29. The method of Statement 24, further comprising receiving a modification to the narrative from the individual member.
Statement 30. The method of Statement 24, further comprising sharing the narrative on the DCN.
Statement 31. The method of Statement 24, wherein the method is used to slow the progression of an adverse health condition of the individual member.
Statement 32. The method of Statement 24, wherein a plot of the narrative comprises a theme of overcoming a challenge.
Statement 33. The method of Statement 24, wherein the message comprises at least one of a suggestion of a behavior change that matches the at least one value or a post is posted in the DCN related to a behavior change that matches the at least one value.
Statement 34. The method of Statement 24, wherein the message comprises a request to the individual member to reflect on at least one of their lifestyle, adverse health condition, environment, and combinations thereof.
Statement 35. The method of Statement 24, wherein the method is used to manage a chronic disease or lower a cost of health care in the population.
Statement 36. The method of Statement 24, further comprising determining at least one value from the narrative, and wherein the message is based on the narrative and the at least one value.
Statement 37. A method to optimize at least one question for an individual member, the method comprising: providing a digital communication network (DCN) for members of the population to communicate with each other; transmitting at least one question to the individual member of the population and a request for the individual member to answer the at least one question; receiving at least one answer to the at least one question from the individual member; automatically analyzing the at least one answer to determine if the at least one answer meets a suitability threshold; and repeating transmitting the at least one question, receiving the at least one answer, and automatically analyzing the at least one answer when the at least one answer does not meet the suitability threshold, wherein the suitability threshold corresponds to measures of at least one of a future outlook, concreteness of a view point, emotional valence, and combinations thereof.
Statement 38. The method of Statement 37, wherein a combination of all of the at least one answers received are used to determine a suitability of the at least one question.
Statement 39. The method of Statement 37, wherein the at least one question comprises a plurality of questions.
Statement 40. The method of Statement 37, wherein the at least one question is selected from a pool of questions designed to illicit a suitable response.
Statement 41. The method of Statement 37, wherein one or more attributes of a subsequent question is different to one or more attributes of the at least one question.
Statement 42. The method of Statement 39, wherein the plurality of questions are asked in a single session.
Statement 43. The method of Statement 38, wherein the plurality of questions are asked in a plurality of separate sessions, and wherein each session is separated by a period of time.
Statement 44. The method of Statement 43, wherein the period of time is at least one of an hour, a day, or a week.
Statement 45. The method of Statement 43, further comprising requesting that the individual member reflect on the at least one answer prior to a subsequent session.
Statement 46. The method of Statement 37, wherein the at least one question that meets the suitability threshold are used to determine at least value of the individual member.
Statement 47. The method of Statement 37, wherein the at least one question that meets the suitability threshold are used to generate a narrative about at least one of the individual member’s life state, current lifestyle, the journey that created the narrative, aspirations for the future, and at least one value.
Statement 48. The method of Statement 37, wherein the at least one question and the at least one answer are transmitted and received at least one of verbally or in text.
Statement 49. A method for optimizing one or more questions, the method comprising: providing a digital communication network (DCN) for members of the population to communicate with each other; transmitting at least one question to the individual member of the population and a request for the individual member to answer the at least one question; receiving at least one answer to the at least one question from the individual member; analyzing the at least one answer to generate an outcome; and transmitting a message to the individual member based on the outcome, wherein the outcome comprises at least one of the individual member’s values, future aspirations, or a narrative.
Statement 50. The method of Statement 49, further comprising: transmitting at least one subsequent question; receiving at least one subsequent answer; analyzing the at least one subsequent answer to determine a subsequent outcome; measuring a similarity between the outcome and the subsequent outcome; and repeating transmitting the at least one subsequent question, receiving the at least one subsequent answer, determining the subsequent outcome, and measuring the similarity when the similarity is below a similarity threshold, wherein the similarity threshold assess a difference between one or more parameters of the outcome and one or more parameters of the subsequent outcome.
Statement 51. The method of Statement 50, wherein a combination of all of the at least one answers received are used to generate the outcome.
Statement 52. The method of Statement 51, wherein the at least one question is asked before the outcome is generated.
Statement 53. The method of Statement 51, wherein similarity is requested for two subsequent question of the at least one subsequent question to satisfy the similarity threshold.
Statement 54. A method for inducing reflection in an individual member of a population, the method comprising: providing a digital communication network (DCN) for members of the population to communicate with each other; transmitting at least one question to the individual member of the population and a request for the individual member to answer the at least one question; receiving at least one answer to the at least one question from the individual member; and automatically analyzing the at least one answer to determine an emotional valence of the individual member, wherein a subsequent question is based in part on increasing the emotional valence of subsequent answers.

What is claimed is:

1. A method for identifying at least one value of an individual member of a population, the method comprising:
providing a digital communication network (DCN) for members of the population to communicate with each other;
transmitting at least one question to the individual member of the population and a request for the individual member to answer the at least one question, wherein the at least one question does not solicit the individual member to provide the at least one value;
receiving at least one answer to the at least one question from the individual member;
automatically analyzing the at least one answer to determine the at least one value held by the individual member; and
transmitting a message to the individual member based on the at least one value determined by the analysis of the at least one answer.

2. The method of claim 1, wherein the at least one answer is analyzed using a large language model to assess the at least one value contained within the answer.

3. The method of claim 1, wherein the at least one value comprises at least one of trust, interpersonal harmony, care for long-term outcomes, and family continuity.

4. The method of claim 1, wherein the at least one value determined is communicated to the individual member through the DCN and a request to reflect on the communicated values is transmitted to the individual member.

5. The method of claim 1, wherein the message comprises at least one of a suggestion of a behavior change that matches the at least one value or a post is posted in the DCN related to a behavior change that matches the at least one value.

6. The method of claim 1, wherein the method is used to understand a motivation for a change in an individual member.

7. The method of claim 1, wherein the method is used to at least one of manage a chronic disease or lower a cost of health care in the population or slow the progression of an adverse health condition in the individual member.

8. The method of claim 1, wherein the message comprises a communication aligned with an adverse condition associated with the individual member, wherein the adverse condition is determined by automatically analyzing communications from the individual member in the DCN, and wherein the adverse condition comprises at least one of chronic systemic inflammation, malaise, low energy, a disease, a health risk, social dysfunction, or a prodromal disease.

9. The method of claim 8, wherein the disease comprises at least one of obesity, diabetes, rheumatoid arthritis, Crohn’s disease, psoriasis, eczema, cardiovascular disease, congestive heart failure, chronic obstructive pulmonary disease, asthma, depression, or anxiety, and wherein the health risk is at least one of falling or becoming infected with an illness.

10. A method for generating a narrative for an individual member of a population, the method comprising:
providing a digital communication network (DCN) for members of the population to communicate with each other;
transmitting at least one question to the individual member of the population and a request for the individual member to answer the at least one question, wherein the at least one question does not solicit the individual member to provide a narrative of the individual member’s history, aspiration, and at least one value;
receiving at least one answer to the at least one question from the individual member;
automatically analyzing the at least one answer to generate the narrative about the individual member; and
transmitting a message to the individual member based on the narrative determined by the analysis of the at least one answer.

11. The method of claim 10, wherein the individual member is the protagonist of the narrative.

12. The method of claim 10, wherein the narrative comprises elements that reflect at least one of the individual’s life state, current lifestyle, the journey that created the narrative, the individual member’s aspirations for the future, and the individual member’s values.

13. The method of claim 10, further comprising receiving a modification to the narrative from the individual member.

14. The method of claim 10, wherein a plot of the narrative comprises a theme of overcoming a challenge.

15. The method of claim 10, wherein the message comprises at least one of a suggestion of a behavior change that matches the at least one value or a post is posted in the DCN related to a behavior change that matches the at least one value.

16. A method to optimize at least one question for an individual member, the method comprising:
providing a digital communication network (DCN) for members of the population to communicate with each other;
transmitting at least one question to the individual member of the population and a request for the individual member to answer the at least one question,
receiving at least one answer to the at least one question from the individual member;
automatically analyzing the at least one answer to determine if the at least one answer meets a suitability threshold; and
repeating transmitting the at least one question, receiving the at least one answer, and automatically analyzing the at least one answer when the at least one answer does not meet the suitability threshold,
wherein the suitability threshold corresponds to measures of at least one of a future outlook, concreteness of a view point, emotional valence, and combinations thereof.

17. The method of claim 16, wherein a combination of all of the at least one answers received are used to determine a suitability of the at least one question.

18. The method of claim 16, wherein one or more attributes of a subsequent question is different to one or more attributes of the at least one question.

19. The method of claim 16, wherein the at least one question that meets the suitability threshold are used to determine at least a value of the individual member.

20. The method of claim 19, wherein the at least one question that meets the suitability threshold are used to generate a narrative about at least one of the individual member’s life state, current lifestyle, the journey that created the narrative, aspirations for the future, and at least one value.
